(19) United States
(12) Patent Application Publication
(10) Pub. No.: US 2012/0216803 A1
(43) Pub. Date: Aug. 30, 2012

(54) DEVICE FOR SECURING AIRWAY TUBING TO A PATIENT

(75) Inventor: Yakov Trodler, Lod (IL)

(73) Assignee: TRODEK LTD., Lod (IL)

(21) Appl. No.: 13/446,547

(22) Filed: Apr. 13, 2012

Related U.S. Application Data
(63) Continuation-in-part of application No. 12/067,459, filed on Mar. 20, 2008, now Pat. No. 8,156,934.
(60) Provisional application No. 60/596,404, filed on Sep. 21, 2005.

(51) Int. Cl. A61M 16/04

(52) U.S. Cl. 128/200.26

(57) ABSTRACT

An airway tube securing device (ATSD) having a tubular constrictor attached to an oral piece and a fastener for fastening the ATSD to the face of a patient provides for the correct placing and fixation of an airway tube within the patient's airways. A rotatable clamp provides for constricting the airway tube thereby securing it to the oral piece. An optional mouthpiece attached to the proximal face of the oral piece provides for the protection of the tube from the jaws of a patient. One or two apertures in the oral piece provide for further insertion of tubing, auxiliary intubation tools and or probes.

### CROSS REFERENCE TO RELATED APPLICATIONS

This application is a continuation-in-part of U.S. patent application Ser. No. 12/067,459, filed Mar. 20, 2008, now U.S. Pat. No. 8,156,934, which is a national stage application of PCT/IL2006/001116, filed Sep. 21, 2006, which claims priority to U.S. Provisional Patent Application Ser. No. 60/596,404, filed Sep. 21, 2005, the disclosures of which are hereby expressly incorporated by reference herein in their entirety.

### FIELD OF THE DISCLOSURE

The present disclosure relates to auxiliary tools for securing airway tubing to a patient.

### BACKGROUND OF THE DISCLOSURE

Oral intubation is commonly applied in the course of medical treatments such as operations involving anesthesia, in which breathing is assisted and controlled by airway tubing. Endotracheal and/or laryngeal tubes are typically used in such operations for delivering oxygen and medicines to the trachea. The correct placement and fixation of such tubes is critical in maintaining the patient's wellbeing. However, displacement of such tubes remains a risk. Even initially as an endotracheal and/or laryngeal tube is properly positioned and secured by currently available means, the tube will often displace due to mechanical activity, associated with the instrumentation and patient movements. Such displacement can harm the patient in several ways. Effort invested in the development of superior intubation means, yielded auxiliary intubation tools, aids and procedures. Securing the insertion of endotracheal and/or laryngeal tubes still require attention of the medical team which interfere with other essential procedures. A device disclosed in U.S. Pat. No. 5,626,128 addresses this problem by maintaining an endotracheal tube in proper position within a patient's mouth by means of an oral adhesive composition. However, the employment of such a device obviates the use of some additional devices targeted at the oral cavity, such as suction tubes.

### SUMMARY

The present disclosure provides an airway tube securing device (ATSD) having a tubular constrictor attached to an oral piece and a fastener for fastening the ATSD to the face of a patient to provide for the correct placing and fixation of an airway tube within the patient's airways.

According to an embodiment of the present disclosure, a device is provided for securing an airway tube in a patient. The device includes an oral piece configured to couple to the patient, a constrictor extending longitudinally from the oral piece, the constrictor comprising a plurality of segments that are radially-spaced apart, the plurality of segments cooperating to define an aperture for receiving the airway tube, each segment increasing in thickness from a first side to a second side, and a clamp rotatably coupled to the constrictor to adjust the constrictor between a first configuration and a second configuration, the clamp comprising a plurality of projections that are radially-spaced apart and that protrude inwardly toward the constrictor, the projections of the clamp positioned near the first sides of the segments in the first configuration and positioned near the second sides of the segments in the second configuration, the segments extending further inwardly toward the airway tube in the second configuration than the first configuration.

According to another embodiment of the present disclosure, a device is provided for securing an airway tube in a patient. The device includes an oral piece configured to couple to the patient, a constrictor extending longitudinally from the oral piece, the constrictor comprising a plurality of segments that are radially-spaced apart, the plurality of segments cooperating to define an aperture for receiving the airway tube, and a clamp comprising a plurality of projections that are radially-spaced apart and that protrude inwardly toward the constrictor, the clamp rotating relative to the constrictor to adjust the constrictor between a first configuration and a second configuration, the constrictor applying a clamping force to the airway tube that increases continuously from the first configuration to the second configuration.

According to yet another embodiment of the present disclosure, a method is provided for securing an airway tube in a patient. The method includes the steps of providing a device that includes an oral piece, a constrictor coupled to the oral piece, the constrictor having a plurality of radially-spaced apart segments that cooperate to define an aperture, and a clamp, the clamp having a plurality of radially-spaced apart projections, securing the oral piece to the patient, inserting the airway tube into the aperture of the constrictor, and rotating the clamp around the constrictor to continuously tighten the segments of the constrictor against the airway tube.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a side view of a preferred embodiment of the airway tube securing device (ATSD) according to the present disclosure;

FIG. 2 is a side view of the ATSD shown in FIG. 1, with an elastic facial fastening strip;

FIG. 3 is a top side view of the ATSD shown in FIG. 1;

FIG. 4 is an isometric view of a preferred embodiment of the constrictor according to the present disclosure;

FIG. 5A is an isometric view of an ATSD according to the present disclosure, attached to a Murphy cuffed endotracheal tube;

FIG. 5B is an isometric view of an ATSD according to the present disclosure, attached to a laryngeal mask;

FIG. 6 is an isometric view of an ATSD according to another preferred embodiment of the present disclosure;

FIG. 7 is a sectional view of a constrictor according to another preferred embodiment of the present disclosure;

FIG. 8 is an isometric view of an ATSD according to another preferred embodiment of the present disclosure;

FIG. 9 is an isometric view of an ATSD according to yet another preferred embodiment of the present disclosure, the ATSD shown with a fastening strip;

FIG. 9A is a detailed isometric view of the portion of the ATSD circled in FIG. 9;

FIG. 10 is an isometric view of the ATSD of FIG. 9 shown without a rotatable clamping means;

FIG. 10A is a detailed isometric view of the portion of the ATSD circled in FIG. 10;

FIGS. 11A and 11B are isometric views of the rotatable clamping means of FIG. 9;

FIG. 12A is a cross-sectional view of the ATSD of FIG. 9A, taken along line 12-12 of FIG. 9A, and showing the ATSD in an open state;

FIG. 12B is a cross-sectional view similar to FIG. 12A, showing the ATSD in a constricted state;

FIG. 13A is a top plan view of the ATSD in the open state of FIG. 12A; and

FIG. 13B is a top plan view of the ATSD in the constricted state of FIG. 13A.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

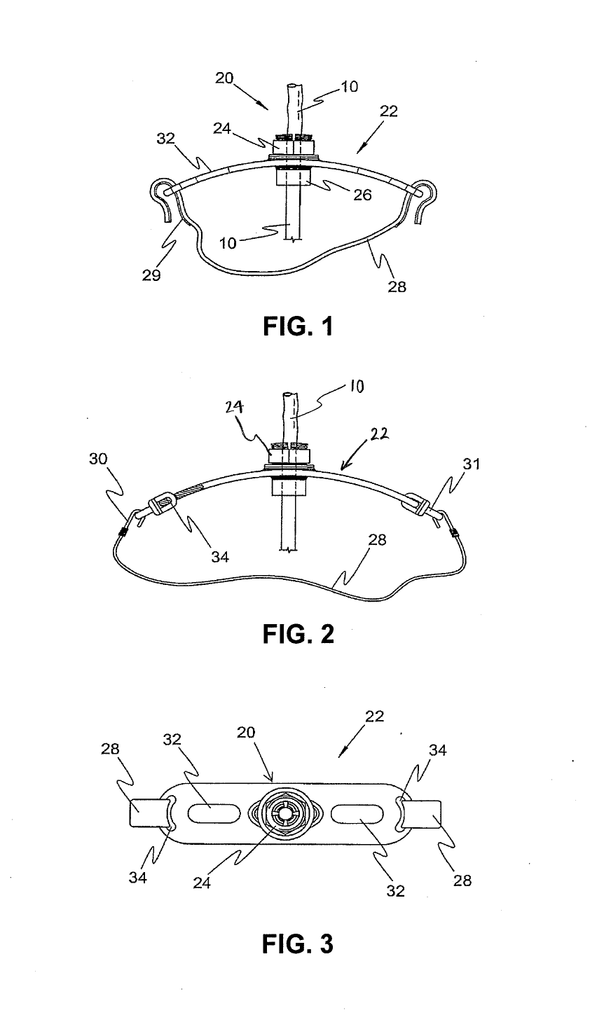

Reference is first made to FIG. 1, which schematically shows a side view of an airway tube securing device (ATSD), according to a preferred embodiment of the present disclosure. A section of airway tube 10 is shown traversing a pass-through aperture in segmented constrictor 20 of oral piece 22. By rotating clamping means 24 the inner diameter of the segmented constrictor 20 diminishes, thereby clamping airway tube 10 and securing it firmly to oral piece 22. Mouthpiece 26, which is substantially coaxial with airway tube 10, protrudes from the proximal face of oral piece 22. The mouthpiece 26 is inserted in a patient's oral cavity between upper and lower jaws. Face fastening strip 28 with Velcro® strips 29 attached to both ends, respectively, fastens the oral piece 22 to the patient's face by strapping fastening strip 28 against the patient's neck onto either its proximal or distal faces. Other variants of the fastening mechanism include different connecting means, used to connect strips 29 to oral piece 22, including among other possibilities buckles, clamping connectors, hooks attached to the strips' ends, or Velcro® strips attached to the proximal face of the fastening strip.

Reference is now made to FIG. 2, showing an oral piece 22 of the disclosure, implementing an elastic facial fastening strip 28, using hook 30 inserted into ring 31 connected to the oral piece 22 end by means of strip connector aperture 34. Strip hooks 30 may also be directly inserted into strip connector apertures 34. Hook 30 attached at the end of the fastening strip 28 is shown passing through strip connector apertures 34.

Reference is made to FIG. 3 showing a topside view of an ATSD of the disclosure. Segmented constrictor 20 is located at the center of oral piece 22. Two additional apertures 32, adjacent segmented constrictor 20 and distributed symmetrically at its sides, facilitate the application of additional devices such as thermometers, probes and/or suction tubes. The ends of fastening strip 28 are shown passing through strip connector apertures 34 symmetrically located on both sides of the oral piece 22.

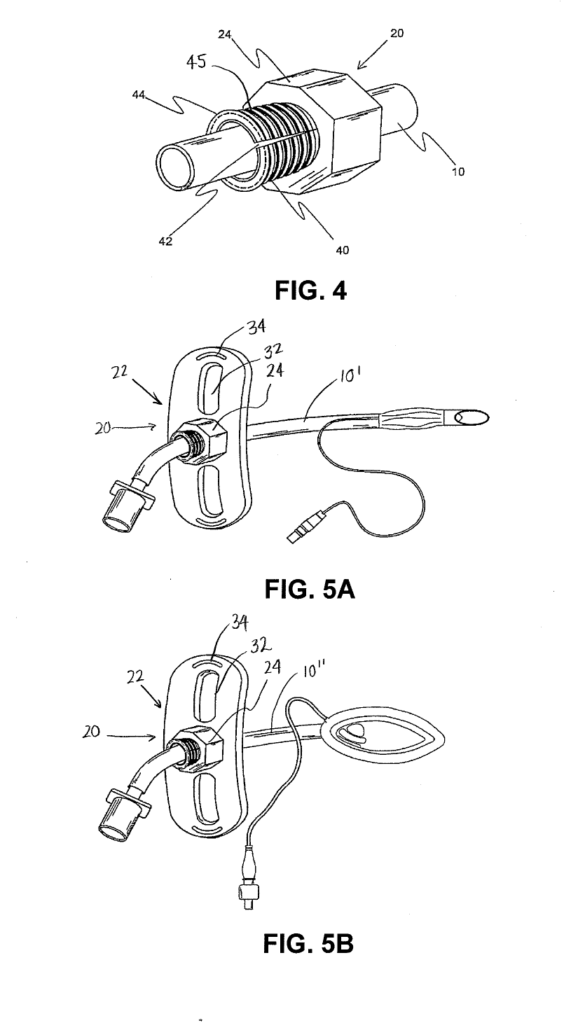

Reference is made to FIG. 4 showing an isometric view of segmented constrictor 20, in which airway tube 10 is inserted. Set of constricting segments 40, having a cylindrical thread 45 on their external surfaces and a conic interior (not shown) is split by longitudinal section 42 into two segments. One end of each of the constricting segments 40 is firmly secured to the oral piece 22. The internal surface of constricting segments 40 is roughened for higher friction and improved clamping of the airway tube 10. The radius of the lumen of segmented constrictor 20 decreases as the distance from the distal face of the oral piece 22 increases. Insertion of an airway tube 10 into segmented constrictor 20 divides the longitudinal constricting segments 40 along longitudinal section 42. Segmented constrictor 20 according to the disclosure also consists of a clamping means 24 that is a hollow body such as a nut, rotatable over the constricting segments 40 such that the pressing force exerted over the constricting segments 40 increases or decreases according to its direction of rotation. Rotating clamping means 24 clockwise changes the inner diameter of the segmented constrictor 20 due to its interior conical shape. Decreasing the inner diameter of segmented constrictor 20 applies pressure to the exterior surface of airway tube 10 pressing it against the constricting segments 40. Flexible ring 44 attached to one or both ends of the segmented constrictor wall 40 prevents clamping means 24 from falling off.

By means of properly selecting internal conical shape and angle formed by the inner surface of the constricting segments 40 and external thread spacing 45, the same segmented constrictor 20 can be adapted to a range of tube diameters starting with one of 2.5 millimetres (mm) suitable for neonates and up to 10-11 mm for male adults. Rotating the constrictor nut 24 by a specific rotational angle changes the inner diameter of the segmented constrictor 20 proportionally. One segmented constrictor 20 may be adapted to cover the entire range of available airway tubes 10.

Securing the airway tube 10 to the oral piece 22 is typically achieved at a point in which tightening the segmented constrictor nut 24 requires a significantly higher rotational torque. An experienced operator is capable of securing the airway tube 10 fixed to the oral piece 22 by sense of touch. It is possible to ensure that the suitable rotational angle of the nut 24 for loosening or tight clamping of a specified airway tube 10 is easily recognized by the medical team. One way, for example, is to configure the segmented constrictor 20 such that rotating the constrictor nut 24 by 90 degrees is equivalent to a 0.5 mm change in the diameter of the airway tube 10. Alternatively, it is possible to apply color to the thread coils 45 accordingly, employing different hues for designating the progression of the constricting nut 24 on the thread 45. Namely, threads coils 45 corresponding to tube diameter below 3 mm are colored with black, the range 3-4 mm with deep red, 4-5 light red, 5-6 orange, 6-7 yellow, 7-8 green 8-9 blue, 9-10 violet and above 10 white. Other variants consist of a few dedicated oral pieces 22 having segmented constrictors 20 corresponding to different ranges of airway tube diameters and further employing color coding corresponding to these different ranges, in a similar scheme as described above.

In another embodiment of the present disclosure, a knob is employed, such that distinctly audible clicking sounds indicate a proper alignment with the inserted tube 10. The knob is installed at the segmented constrictor 20 side extending sideways at the distal side of the oral piece 22. The knob is coupled to the constrictor nut 24 by means of an axle. Click indications correspond to the following tube diameter values: 2.5, 3, 3.5, and 4 mm.

The ATSD in accordance with the present disclosure is typically made of plastic material that can sustain high temperatures. Such an ATSD can be sterilized and reused, thus providing an inexpensive and valuable intubation aid to be used in the operation theatre. The ATSD is accommodated for attaching various laryngeal as well as endotracheal airway tubes in place. Reference is made to FIGS. 5A-B. In FIG. 5A an ATSD applied to a Murphy cuffed endotracheal tube 10′ is shown. In FIG. 5B an ATSD as applied to a laryngeal mask 10″ is shown. A suitable ATSD may be packaged separately or together with a respective endotracheal airway tube 10′, or with a laryngeal mask 10″, as an airway intubation kit, ready for use by the medical staff

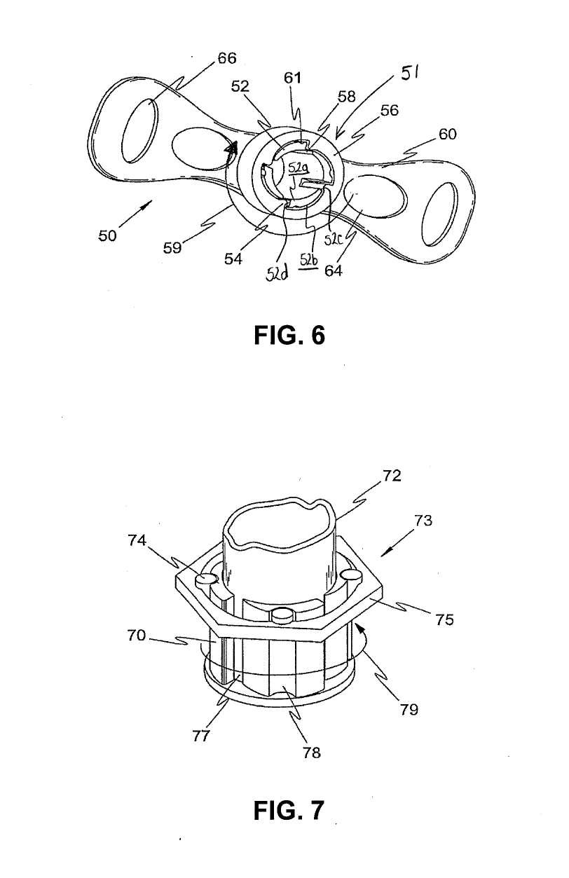

Reference is now made to FIG. 6, which shows an ATSD 50 according to another preferred embodiment of the disclosure. ATSD 50 of FIG. 6 includes a segmented constrictor 51 having a longitudinally segmented tube, the wall of which is divided into set of constricting segments 52 separated by recesses 58. ATSD 50 also includes a rotatable clamping means 56 around the segmented constrictor 51.

Each constricting segment 52 has an inner surface 52a that faces the airway tube (not shown), an outer surface 52b that faces clamping means 56, a first side 52c, and an opposite second side 52d. Each constricting segment 52 has a tapering flank. In the illustrated embodiment of FIG. 6, for example, outer surface 52b of each constricting segment 52 ramps inwardly from second side 52d to first side 52c, such that each constricting segment 52 tapers inwardly from second side 52d to first side 52c. This tapering effect is evident if FIG. 6, because each constricting segment 52 is generally wedge-shaped in cross-section, having more thickness at second side 52d than at first side 52c,

The segmented constrictor 51 of ATSD 50 is shown in an open or unconstricted state in FIG. 6, where the inwardly protruding projections 54 located on the inner surface of annular clamping means 56 are projected into corresponding recesses 58 spacing constricting segments 52 apart. The airway tube (not shown) is securely locked inside oral piece 60 by rotating clamping means 56 in the direction indicated by arrow 59. More specifically, the airway tube is locked by rotating projections 54 of clamping means 56 across the ramped outer surfaces 52b of constricting segments 52 from first sides 52c toward second sides 52d. By such rotation, projections 54 force inner surfaces 52a of constricting segments 52 against the exterior surface of the airway tube. Because constricting segments 52 grow in thickness from first sides 52c to second sides 52d, the clamping force that is transferred from clamping means 56 to constricting segments 52 and from constricting segments 52 to the airway tube may increase continuously as projections 54 of clamping means 56 travel from first sides 52c toward second sides 52d of constricting segments 52.

Clamping means 56 is securely locked in a constricting position, when projections 54 reach corresponding recesses 61 in outer surfaces 52b of constricting segments 52. In FIG. 6, recesses 61 are located near second sides 52d of constricting segments 52. Thus, the clamping force that is transferred from clamping means 56 to constricting segments 52 and from constricting segments 52 to the airway tube may increase continuously until projections 54 enter recesses 61 near second sides 52d of constricting segments 52. The airway tube is unlocked by rotating clamping means 56 in the opposite direction of arrow 59 to release projections 52 from recesses 61. Depending on the size of the airway tube therein, segmented constrictor 51 may achieve a desired clamping force against the airway tube without rotating clamping means 56 all the way to recesses 61. Thus, clamping means 56 may reach its final position somewhere between first sides 52c and second sides 52d of constricting segments 52.

Oral piece 60 has one or two additional apertures 64 for the optional insertion of additional devices and/or tubes as described hereinbefore. Strip connector apertures 66 provide for connecting a fastening strip to oral piece 60. Oral piece 60 has the option for a mouthpiece attachment to its proximal face, providing for protection of the airway tubes from the patient's jaws.

Reference is now made to FIG. 7, which shows a sectional view of a segmented constrictor 73 according to another embodiment of the present disclosure. The segmented constrictor 73 is shown in a constricting position in FIG. 7. Constricting segments 70 of segmented constrictor 73 compress airway tube 72 as pawls 74 of annular clamping means 75 press constricting segments 70 against the wall of airway tube 72. By such pressing the lumen of segmented constrictor 73 becomes partially conic as its diameter decreases along the flanks of the constricting segments 70 as the distance from the distal face of the oral piece (not shown) increases. Segmented constrictor 73 attains a secure constricting position as pawls 74 are shifted from the longitudinal slits 77 between adjacent constricting segments 70 and into external recesses 78 in the constricting segments 70 by rotating clamping means 75 having internal recesses for securing the pawls in the direction indicated by arrow 79.

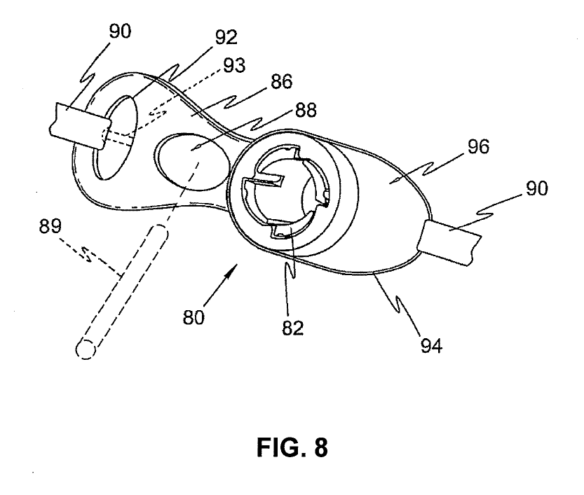

Reference is now made to FIG. 8, in which an ATSD according to another preferred embodiment is shown. This ATSD consists of the same segmented constrictor 80 as the ATSD shown in FIG. 6. However, the oral piece 86 of this ATSD has only one side having an optional aperture 88. This aperture 88 provides for the insertion of additional devices 89 (shown in phantom), where each device 89 may be in the form of a thermometer, an ultrasound probe, or a suction tube, for example. Fastening strip 90 is connected to corresponding aperture 92 located at the end of oral piece 86. Optionally, oral piece 86 includes a buckle 93 (shown in phantom) for connecting the ATSD to fastening strip 90. Noose 94 attached to the other end of fastening strip 90 is hooked to segmented constrictor 80 such that a space 96 is made available for the insertion of additional devices and/or probes (such as an ultrasound probe) into a patient's oral cavity.

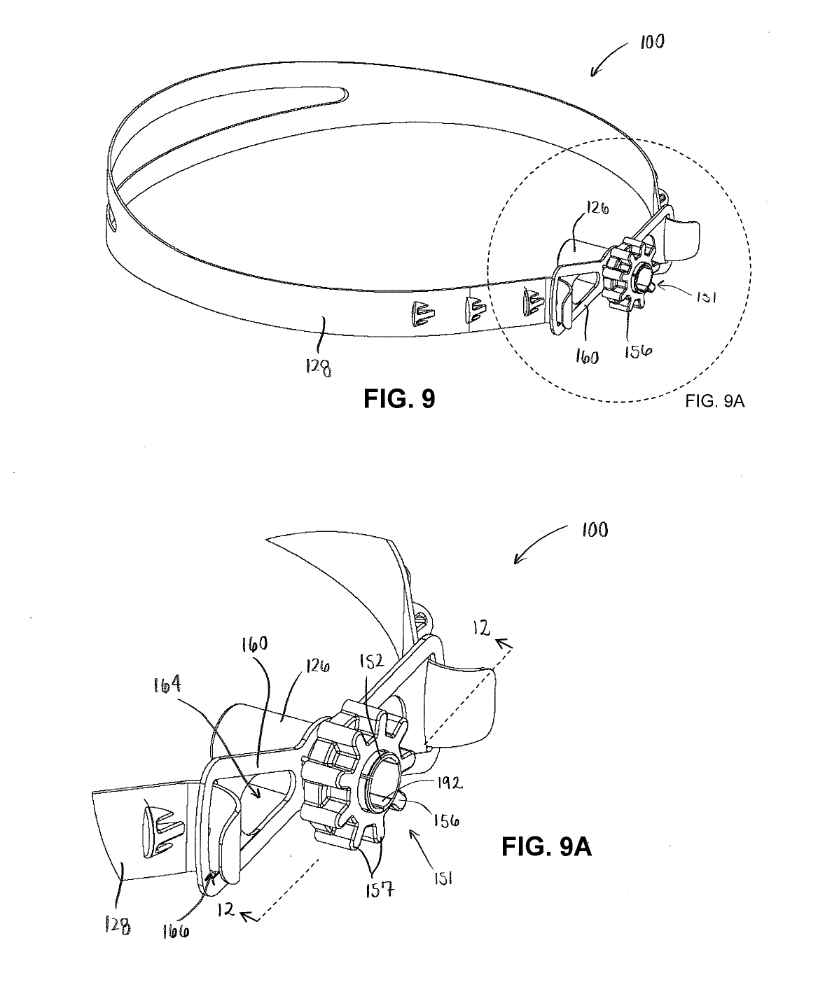

Reference is now made to FIGS. 9-13B, which show ATSD 100 according to another preferred embodiment of the disclosure. ATSD 100 is similar to the above-described ATSD's, especially ATSD 50 of FIG. 6, with like reference numerals indicating like elements.

ATSD 100 includes oral piece 160. Oral piece 160 includes additional side apertures 164 for the optional insertion of additional devices and/or tubes, as described hereinbefore. Oral piece 160 also includes strip connector apertures 166 for connecting a fastening strip 128 to oral piece 160, as shown in FIG. 9. Oral piece 160 further includes mouthpiece 126, as shown in FIG. 9, that is inserted in a patient's oral cavity between upper and lower jaws to protect the airway tube.

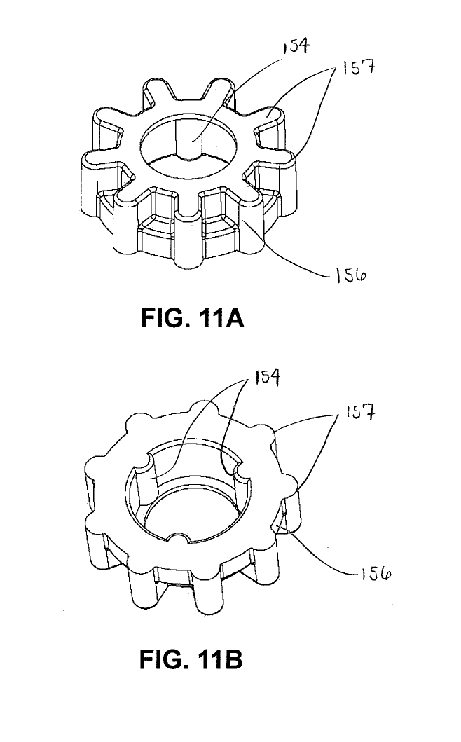

ATSD 100 also includes segmented constrictor 151, which includes longitudinal constricting segments 152 separated by recesses 158. ATSD also includes a rotatable clamping means 156, which is shown separately in FIGS. 11A and 11B. The illustrative clamping means 156 includes a plurality of radially-spaced internal protrusions 154 to operate segmented constrictor 151, as well as a plurality of radially-spaced external protrusions 157 to facilitate gripping and turning by a user.

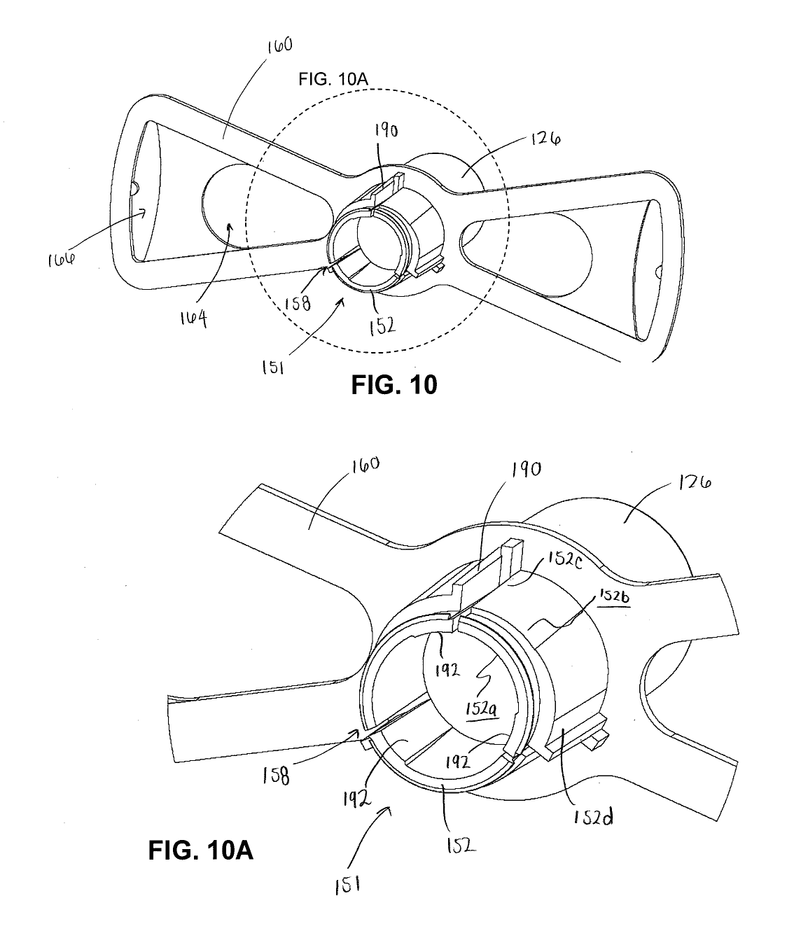

As shown in FIGS. 10 and 10A, each constricting segment 152 has an inner surface 152a that faces the airway tube (not shown), an outer surface 152b that faces clamping means 156, a first side 152c, and a second side 152d. Each constricting segment 152 has a tapering flank. In the illustrated embodiment of FIG. 10, for example, outer surface 152b of each constricting segment 152 ramps inwardly from second side 152d to first side 152c, such that each constricting segment 152 tapers inwardly from second side 152d to first side 152c. This tapering effect is evident if FIG. 10, because each constricting segment 152 is generally wedge-shaped in cross-section, having more thickness at second side 152d than at first side 152c.

Inner surface 152a of each constricting segment 152 may also contribute to this tapering effect. In the illustrated embodiment of FIG. 10A, longitudinal ribs 192 extend from inner surface 152a of each constricting segment 152 near second side 152d. Thus, like outer surface 152b, inner surface 152a of each constricting segment 152 increases in thickness from first side 152c to second side 152d. Each longitudinal rib 192 may also increase in thickness away from oral piece 160 and mouthpiece 126.

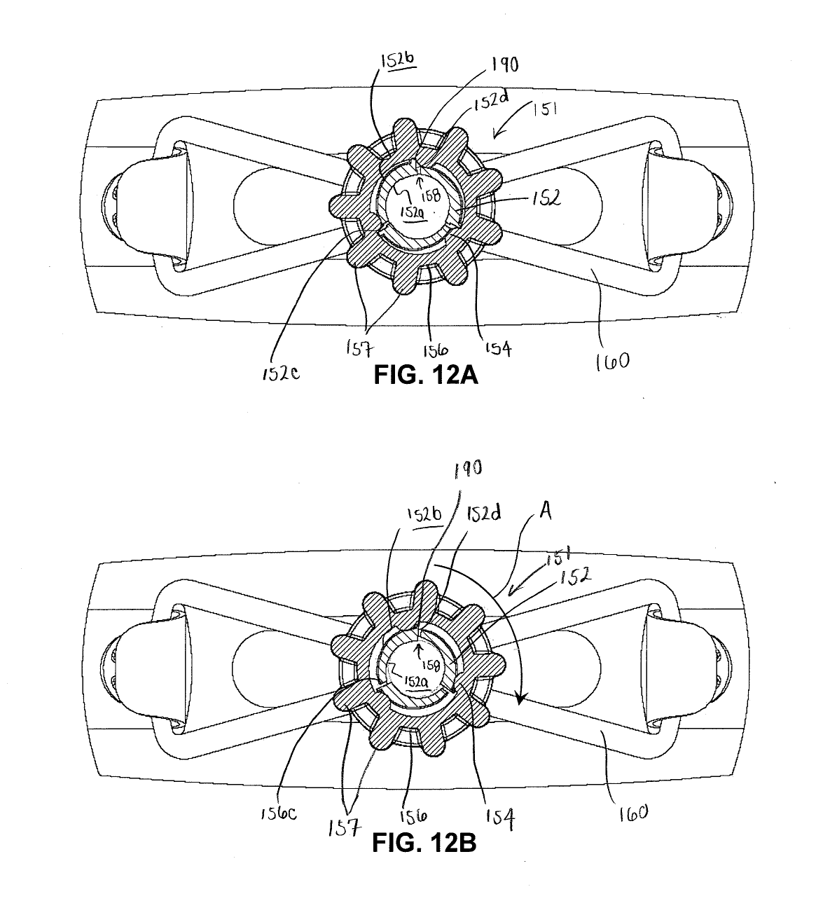

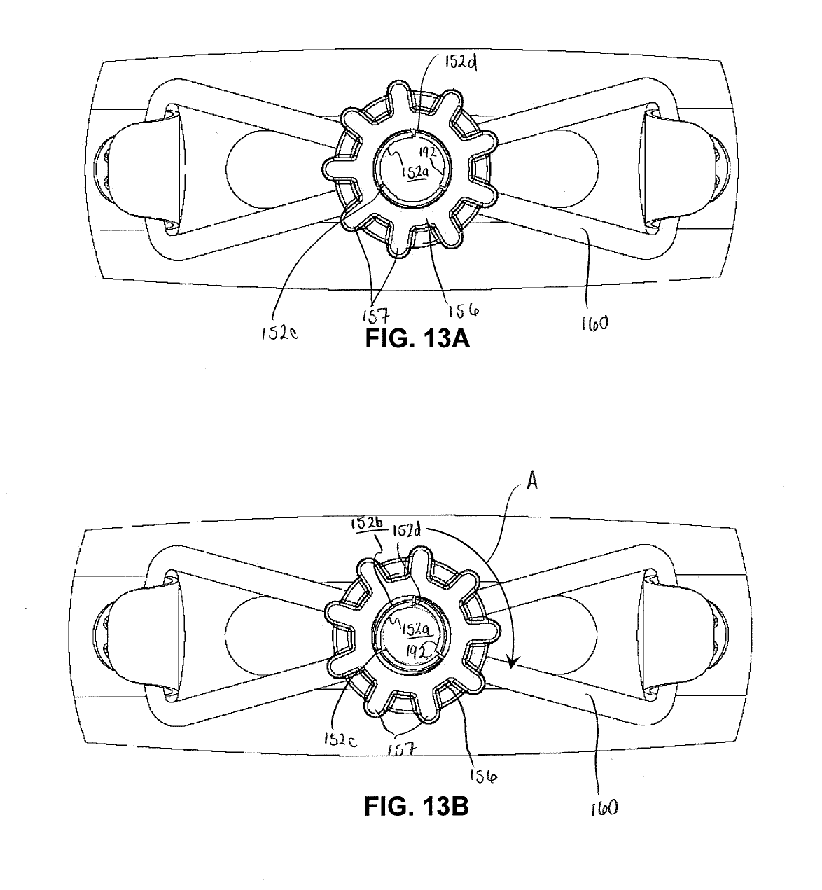

In FIGS. 12A and 13A, segmented constrictor 151 of ATSD 150 is shown in an open or unconstricted state. In this open state, the inwardly protruding projections 154 of clamping means 156 are positioned near first sides 152c of constricting segments 152. Projections 154 of clamping means 156 may be positioned as close as possible to recesses 158 between adjacent constricting segments 152.

Stops 190 on the adjacent constricting segments 152 may prevent projections 154 of clamping means 156 from fully entering and projecting into recesses 158 near first sides 152c when in the open state. Thus, even though FIG. 12A is described as an open or unconstricted state, projections 154 of clamping means 156 may apply some clamping pressure to constricting segments 152 near first sides 152c in the open or unconstricted state.

In FIGS. 12B and 13B, segmented constrictor 151 of ATSD 150 is shown in a clamped or constricted state. From the open state of FIGS. 12A and 13A to the constricted state of FIGS. 12B and 13B, clamping means 156 has been rotated by about 120 degrees in the direction of arrow A. By such rotation, projections 154 of clamping means 156 travel across the ramped outer surfaces 152b of constricting segments 152 from first sides 152c toward second sides 152d. Because constricting segments 152 grow in thickness from first sides 152c to second sides 152d, this rotation of clamping means 156 forces inner surfaces 152a of constricting segments 152 progressively inward toward the airway tube. Thus, the clamping force that is transferred from clamping means 156 to constricting segments 152 and from constricting segments 152 to the airway tube may increase continuously from the open state to the constricted state. Longitudinal ribs 192 on inner surfaces 152a of constricting segments 152 may also contribute to this increasing clamping force as clamping means 156 approaches second sides 152d of constricting segments 152.

Stops 190 on the adjacent constricting segments 152 may prevent projections 154 of clamping means 156 from fully entering and projecting into recesses 158 near second sides 152d when in the clamped state, like in the above-described open state. Depending on the size of the airway tube therein, segmented constrictor 151 may achieve a desired clamping force against the airway tube without rotating clamping means 156 all the way to stops 190. Thus, clamping means 156 may reach its final position somewhere between adjacent stops 190.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A device for securing an airway tube in a patient comprising:
an oral piece configured to couple to the patient;
a constrictor extending longitudinally from the oral piece, the constrictor comprising a plurality of segments that are radially-spaced apart, the plurality of segments cooperating to define an aperture for receiving the airway tube, each segment increasing in thickness from a first side to a second side; and
a clamp rotatably coupled to the constrictor to adjust the constrictor between a first configuration and a second configuration, the clamp comprising a plurality of projections that are radially-spaced apart and that protrude inwardly toward the constrictor, the projections of the clamp positioned near the first sides of the segments in the first configuration and positioned near the second sides of the segments in the second configuration, the segments extending further inwardly toward the airway tube in the second configuration than the first configuration.

2. The device of claim 1, wherein each segment of the constrictor includes an outer surface that faces away from the airway tube and an inner surface that faces toward the airway tube, the outer surface of each segment tapering inwardly from the second side to the first side of the segment.

3. The device of claim 1, wherein the constrictor includes an outer surface that faces away from the airway tube and an inner surface that faces toward the airway tube, the outer surface of the constrictor abutting the projections of the clamp in the second configuration to resist further rotation of the clamp beyond the second configuration.

4. The device of claim 3, wherein the outer surface of the constrictor includes a plurality of recesses that capture the plurality of projections of the clamp in the second configuration to resist further rotation of the clamp beyond the second configuration.

5. The device of claim 3, wherein the outer surface of the constrictor includes a plurality of stops that contact the plurality of projections of the clamp in the second configuration to resist further rotation of the clamp beyond the second configuration.

6. The device of claim 5, wherein the plurality of stops contact the plurality of projections of the clamp in the first configuration to prevent further rotation of the clamp beyond the first configuration.

7. The device of claim 1, wherein a longitudinal recess separates a first segment from an adjacent second segment, the first side of the first segment facing the second side of the second segment across the longitudinal recess, wherein the first side of the first segment is thinner than the second side of the second segment.

8. The device of claim 1, wherein the constrictor permits longitudinal displacement of the airway tube in the first configuration and resists longitudinal displacement of the airway tube in the second configuration.

9. The device of claim 1, wherein the constrictor applies a clamping force to the airway tube that increases continuously from the first configuration to the second configuration.

10. A device for securing an airway tube in a patient comprising:
an oral piece configured to couple to the patient;
a constrictor extending longitudinally from the oral piece, the constrictor comprising a plurality of segments that are radially-spaced apart, the plurality of segments cooperating to define an aperture for receiving the airway tube; and
a clamp comprising a plurality of projections that are radially-spaced apart and that protrude inwardly toward the constrictor, the clamp rotating relative to the constrictor to adjust the constrictor between a first configuration and a second configuration, the constrictor applying a clamping force to the airway tube that increases continuously from the first configuration to the second configuration.

11. The device of claim 10, wherein the clamp rotates by about 120 degrees from the first configuration to the second configuration.

12. The device of claim 10, wherein adjacent segments of the constrictor are separated by a longitudinal recesses, the clamp abutting at least one stop on the constrictor that prevents the plurality of projections on the clamp from entering the longitudinal recesses of the constrictor.

13. The device of claim 10, wherein each segment of the constrictor includes a ramped outer surface.

14. The device of claim 10, wherein each segment of the constrictor is generally wedge-shaped.

15. The device of claim 10, wherein each segment increases in thickness from a first side to a second side, the clamp moving from the first side of each segment in the first configuration toward the second side of each segment in the second configuration.

16. The device of claim 15, wherein each segment of the constrictor includes an inner surface that faces toward the airway tube, a longitudinal rib extending from the inner surface of each segment near the second side.

17. The device of claim 10, wherein the oral piece defines at least one aperture alongside the constrictor that is configured to receive one of a thermometer, a probe, and a suction tube.

18. A method for securing an airway tube in a patient, the method comprising the steps of:
providing a device that includes an oral piece, a constrictor coupled to the oral piece, the constrictor having a plurality of radially-spaced apart segments that cooperate to define an aperture, and a clamp, the clamp having a plurality of radially-spaced apart projections;
securing the oral piece to the patient;
inserting the airway tube into the aperture of the constrictor; and
rotating the clamp around the constrictor to continuously tighten the segments of the constrictor against the airway tube.

19. The method of claim 18, wherein the rotating step comprises moving each projection of the clamp over a single segment of the constrictor.

20. The method of claim 18, wherein the rotating step comprises moving the projections of the clamp over ramped surfaces of the segments.

21. The method of claim 18, wherein the rotating step comprises moving the projections of the clamp over segments of increasing thickness.
